(19) United States
(12) Patent Application Publication
(10) Pub. No.: US 2025/0049826 A1
(43) Pub. Date: Feb. 13, 2025

(54) COMPOSITION FOR PROMOTING THE MATURING OF THE CENTRAL NERVOUS SYSTEM

(71) Applicant: BUONA S.P.A. SOCIETA' BENEFIT, Firenze (IT)

(72) Inventor: Elisabetta ALBI, Perugia (IT)

(73) Assignee: BUONA S.P.A. SOCIETA' BENEFIT, Firenze (IT)

(21) Appl. No.: 18/686,087

(22) PCT Filed: Aug. 23, 2022
(86) PCT No.: PCT/IB2022/057883

§ 371 Date: Feb. 23, 2024

(30) Foreign Application Priority Data
Aug. 27, 2021 (IT) 102021000022427

(51) Int. Cl. A61K 31/688; A61K 31/164; A61K 31/202; A61K 31/355; A61K 31/593; A61K 45/06; A61P 25/00

(52) U.S. Cl. CPC A61K 31/688; A61K 31/164; A61K 31/202; A61K 31/355; A61K 31/593; A61K 45/06; A61P 25/00

(57) ABSTRACT

A composition for promoting the maturing of the nervous system comprising vitamin D3 and sphingomyelin.

### FIELD OF THE INVENTION

The present invention relates to a composition for promoting the maturing of the central nervous system. In particular, the present invention relates to the use of a composition containing vitamin D3 and sphingomyelin to promote, support or optimize the formation and outgrowth of neurites of the nervous system cells.

### BACKGROUND OF THE INVENTION

To date, the use of cholecalciferol (vitamin D3 in the active form) is known to help the bone and muscle function, as described for instance in EP2532351A1 and, immune system efficiency.

In order to perform its functions in an optimised manner, the cholecalciferol needs to be bound to its receptor (VDR), present in lipid microdomains rich in sphingomyelin content.

It is known in the art that dietary compositions, like artificial milk, rich in sphingomyelin and containing vitamin D3 among other components, promote de novo myelination of cells of the central nervous system, see for instance WO2017/102717A1.

However, there are currently no prior art compositions for which the link between cholecalciferol and its receptor (VDR) is optimised in order to amplify the functions of the cholecalciferol, useful in particular to the central nervous system maturation.

The aim of the present invention is therefore to provide a composition for the maturing of the central nervous system that is able to obviate the above-mentioned drawbacks of the prior art.

More specifically, the aim of the present invention is to provide a composition that maximises the effects of the cholecalciferol on the nervous system cell maturation.

These aims and others are achieved by using a composition comprising the technical features described in the appended claims.

### SUMMARY OF THE INVENTION

In a first aspect, the present invention refers to the use of a composition comprising vitamin D3 and sphingomyelin to promote, support or optimize the formation and outgrowth of neurites of the nervous system cells.

In a further aspect, the present invention refers to a method for promoting, supporting or optimizing the formation and outgrowth of neurites of the nervous system cells comprising the step (i) of administering to a mammalian, preferably to a human being, a composition comprising vitamin D3 and sphingomyelin.

In a still further aspect, the present invention refers to composition for oral administration comprising from 400 IU to 5000 IU of vitamin D3 and from 2 to 50 mg of sphingomyelin, wherein the ratio of vitamin D3 to sphingomyelin is equal to or greater than 50:1 (IU/mg). The technical features of the invention, with reference to the above-mentioned aims, are clearly described in the accompanying claims and its advantages are apparent from the detailed description which follows.

### BRIEF DESCRIPTION OF THE DRAWINGS

FIG. 1 shows a magnification of a plurality of hippocampus cells in the reference situation (CTR).

FIG. 2 shows a magnification of a plurality of hippocampus cells treated with a first embodiment of a composition according to the present invention.

FIG. 3 shows a magnification of a plurality of hippocampus cells treated with a second embodiment of a composition according to the present invention.

FIG. 4 shows a magnification of a plurality of hippocampus cells treated with a third embodiment of a composition according to the present invention.

FIG. 5 shows a first diagram relating to the volume of the cells, CTR and treated with the first, second and third embodiment of the composition for use according to the present disclosure.

FIG. 6 shows a second diagram relative to the number of cells with neurites expressed in percentage values, relative to the CTR cells and cells treated with the first, second and third embodiment of the composition for use according to the present disclosure.

FIG. 7 shows a third diagram relative to the length of the neurites of the CTR cells and of cells treated with the first, second and third embodiment of the composition for use according to the present disclosure.

FIG. 8 illustrate the cell viability of cells treated with compositions for use according to the present invention with different ratios vitamin D3/sphingomyelin.

FIG. 9 illustrates the gene expression of NEFH encoding for NF200 protein for cells cultured in presence of compositions for use according to the present disclosure containing vitamin D3 and sphingomyelin in different ratios.

### DETAILED DESCRIPTION OF THE INVENTION

In the present description and the appended claims:
the term “vitamin D3” means vitamin D3 in an active form, i.e. cholecalciferol. 1 IU (International Unit) of vitamin D3 corresponds to 0.025 mcg (micrograms) of vitamin D3;
the term “sphingomyelin” (SM) is used to mean a lipid molecule, belonging to the family of sphingolipids, with a structural and functional role. It is a component of the cell membranes where it regulates the fluidity and permits the formation of functional lipid microdomains that, in the nuclear membranes, act as a platform for the VDR. Also in the nervous system cells this specific action of sphingomyelin was demonstrated, in addition to its function as a component of the myelin sheath;
the term “maturation of the nervous system” comprises the differentiation of immature cells towards mature cells characterised by the modification of the volume and shape of the soma and neurite formation;
the term “medium-chain triglycerides” refers to triglycerides with two or three fatty acids having an aliphatic tail of 6-12 carbon atoms;
the term “Glycerophospholipids” indicates glycerol-based phospholipids, a class of lipids whose molecule has a hydrophilic “head” containing a phosphate group and two hydrophobic “tails” derived from fatty acids, joined by an alcohol residue (usually a glycerol molecule);
the term “ceramide” refers to a family of waxy lipid molecules composed of sphingosine and a fatty acid;
the term “cholesterol” indicates a class of organic molecules called lipids;
the term “vitamin E” indicates a group of eight fat soluble compounds that include four tocopherols and four tocotrienols.

In a first aspect, the present invention refers to the use of a composition comprising vitamin D3 and sphingomyelin to promote, support or optimize the formation and outgrowth of neurites of the nervous system cells, in particular of the central nervous system cells.

Preferably, the composition for use according to the present disclosure comprises from 400 IU to 5000 IU of vitamin D3 and from 2 to 50 mg of sphingomyelin, preferably from 400 IU to 800 IU of vitamin D3 and from 2 to 50 mg of sphingomyelin.

In embodiments, the composition for use according to the present disclosure comprises vitamin D3 and sphingomyelin in a ratio vitamin D3/sphingomyelin equal to or greater than 10:1 (IU/mg), preferably equal to or greater than 50:1 (IU/mg).

Preferably, in the composition for use according to the present disclosure the ratio vitamin D3/sphingomyelin ranges from 10:1 (IU/mg) to 400:1 (IU/mg), preferably from 50:1 (IU/mg) to 400:1 (IU/mg), still more preferably from 150:1 (IU/mg) to 350:1 (IU/mg), still more preferably from 160:1 (IU/mg) to 250:1 (IU/mg).

In a preferred embodiment, in the composition for use according to the present disclosure the ratio vitamin D3/sphingomyelin is 200:1 (IU/mg).

In a particularly preferred embodiment, the composition for use according to the present disclosure comprises 400IU of vitamin D3 and 2 mg of sphingomyelin.

The composition for use according to the present invention is particularly suitable for patients with poor nutrition issues or with greater nutritional requirements, for example infants.

In order to further optimise the beneficial effects of the composition according to the present invention, the composition for use according to the present disclosure further comprises at least one additional component selected from the group consisting of: docosahexaenoic acid (DHA), Vitamin E, medium-chain triglycerides, glycerophospholipids, ceramides, cholesterol and mixtures thereof.

The composition is preferably suitable for oral administration and more preferably in the form of a capsule (including microcapsules), a tablet, a water-soluble powder (including an orosoluble or orodispersible powder), a liquid solution, a hydroalcoholic solution or an edible product, like a dietary supplement.

Advantageously, the composition for use according to the present invention is in the form of a liquid solution. In a further aspect, the present invention refers to a method for promoting, supporting or optimizing the formation and outgrowth of neurites of the nervous system cells, preferably of the central nervous system, comprising the step (i) of administering to a mammalian, preferably to a human being, a composition comprising vitamin D3 and sphingomyelin.

In a preferred embodiment, the step (i) comprises or consist of orally administering the composition comprising vitamin D3 and sphingomyelin.

More preferably, the step (i) comprises or consists of orally administering the composition comprising vitamin D3 and sphingomyelin in a form selected from a capsule, a tablet, a water-soluble powder, a liquid solution, a hydroalcoholic solution and an edible product. Preferably, the step (i) comprises or consists of administering a composition comprising vitamin D3 and sphingomyelin as described in any one of the embodiments disclosed above.

According to a preferred embodiment, the step (i) of method according to the present disclosure comprises or consists of administering to a mammalian, preferably a human being, from 400IU to 5000IU of vitamin D3 and from 2 to 50 mg of sphingomyelin at least once a day.

The present disclosure refers also to a composition for oral administration comprising from 400 IU to 5000 IU of vitamin D3 and from 2 to 50 mg of sphingomyelin, wherein the ratio of vitamin D3 to sphingomyelin is equal to or greater than 50:1 (IU/mg).

In preferred embodiments the composition of the present disclosure is as described in the appended claims.

### Experimental Section

### Microscopy Images and Analysis of Differentiated Cells

Immortalized hippocampal neurons HN9.10e (kind gift of Dr. Kieran Breen, Ninewells Hospital, Dundee, U.K.) were grown in DMEM, supplemented with 10% fetal bovine serum, 2 mM L-glutamine, 100 IU/ml penicillin, 100 g/ml streptomycin and 2.5 g/ml amphotericin B (fungizone). Cells were maintained at 37° C. in a saturating humidity atmosphere containing 95% air 5% CO2.

For the experiments, the cells were seeded at 5×10⁵ cells/5 mL medium concentration. Solution of:
first embodiment: 1 μg cholecalciferol+10 μg sphingomyelin (corresponding to 400IU of vitamin D3 and 2 mg of sphingomyelin);
second embodiment: 2 μg cholecalciferol+20 μg sphingomyelin (corresponding to 800IU of vitamin D3 and 4 mg of sphingomyelin);
third embodiment: 1 μg cholecalciferol+20 μg sphingomyelin (corresponding to 400IU of vitamin D3 and 40 mg of sphingomyelin), dissolved with absolute ethanol (20 μL) as vehicle were added to the cultures.

Only 20 μL of absolute ethanol was added in control sample (CTR).

Cells were cultured for 96 hours. The observations were performed by using inverted microscopy EUROMEX FE 2935 (Papenkamp 206836 BD Arnhem, The Netherlands) equipped with a CMEX 5000 camera system (40× magnification). The morphometrical and branching analysis of differentiated cells were performed by ImageFocus software. Cell volume was measured. Cells that showed neurites with one or more were ramifications counted like “branching positive” whereas cells without any sort of ramification were counted like “branching negative”. “Branching positive” cells were measured as percentage of total cells. After images/pixels calibration, the neurite length was measured beginning from the end of the elongation and ending to its soma formation and outgrowth by following its profile; collaterals were not considered into the measurement.

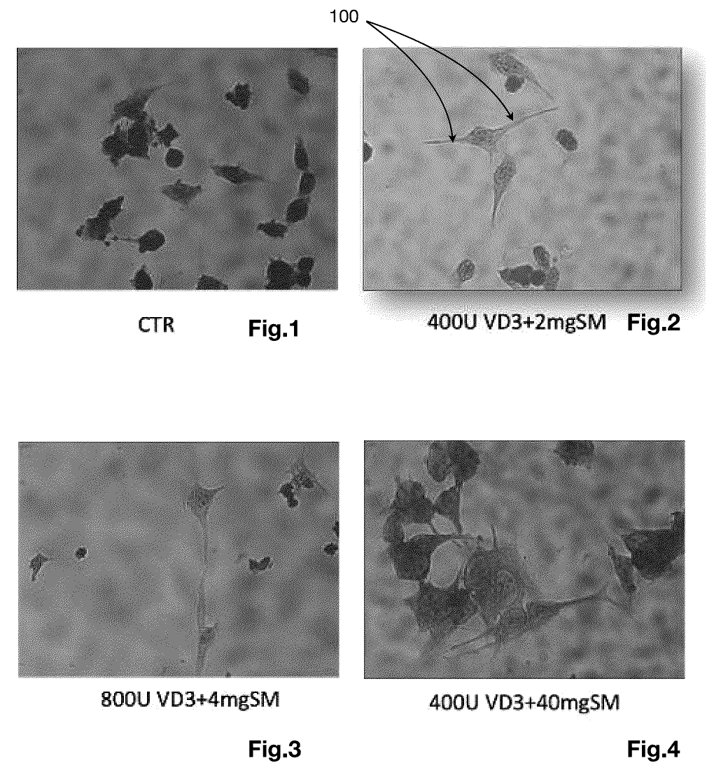

With reference to the accompanying drawings, FIG. 1 shows a magnification of a plurality of hippocampus cells in the reference situation (CTR) without having been treated with the composition according to the present invention.

FIG. 2 shows the magnification of a plurality of hippocampus cells treated with the first embodiment of a composition for use according to the present invention. In the comparison with the CTR situation (FIG. 1) a significant increase in the length of the neurites (100) can be seen as well as a significant increase in the number of cells having at least one developed neurite. This circumstance is shown schematically in FIGS. 6 and 7.

FIG. 3 shows the magnification of a plurality of hippocampus cells treated with the second embodiment of the composition for use according to the present invention.

In the comparison with the CTR situation (FIG. 1) a significant increase in the length of the neurites (100) can be seen as well as a significant increase in the number of cells having at least one developed neurite. This circumstance is shown schematically in FIGS. 6 and 7.

FIG. 4 shows the magnification of a plurality of hippocampus cells treated with the third embodiment of a composition for use according to the present invention.

In the comparison with the CTR situation (FIG. 1) a significant increase in the length of the neurites (100) can be seen as well as a significant increase in the number of cells having at least one developed neurite. This circumstance is shown schematically in FIGS. 6 and 7.

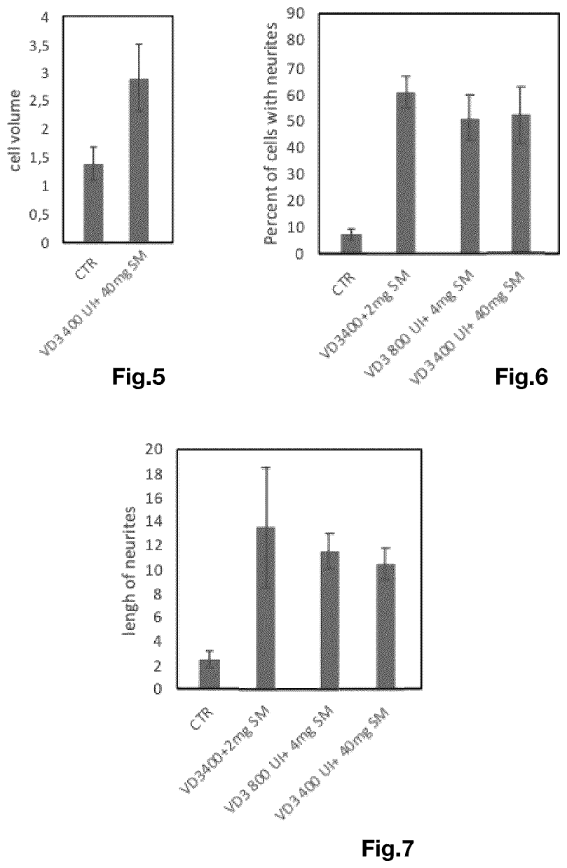

A significant increase in the volume of cells with respect to the reference situation (CTR) is also possible, as shown in FIG. 5.

### Cell Viability

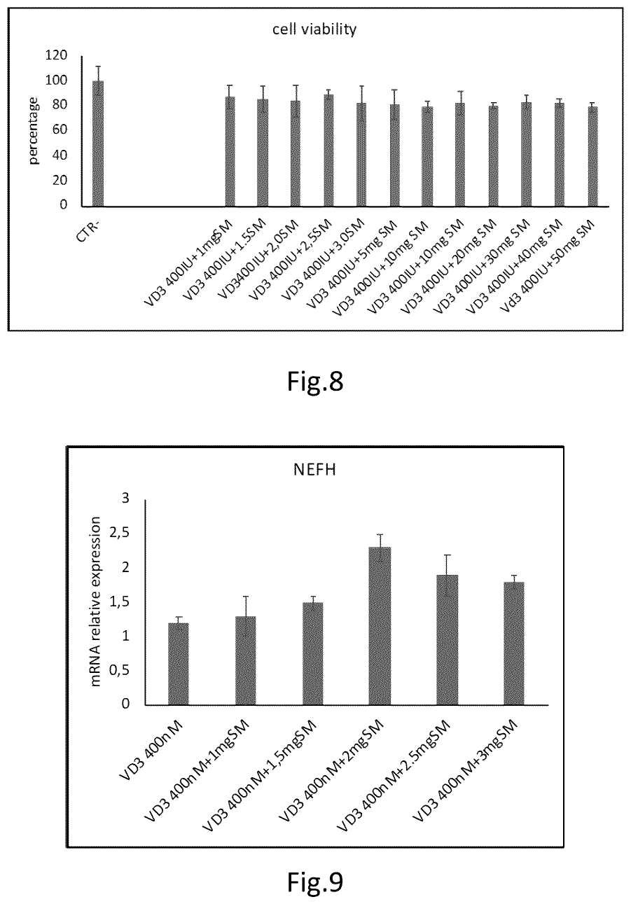

MTT assay was used to test cellular viability of cells treated with the composition for use according to the present invention. HN9.10e cells were seeded into 96-well plates at a density of 1×10⁴ cells/well with DMEM complete medium. After 24 h, culture medium was replaced with fresh, complete medium, and the cells were incubated with increasing concentration of combinations of cholecalciferol and sphingomyelin for 24. Then, MTT reagent was dissolved in PBS 1× and added to the culture at 0.5 mg/mL final concentration. After 3 h incubation at 37° C., the supernatant was carefully removed, and formazan salt crystals were dissolved in 200 μL DMSO that added to each well. The absorbance (OD) values were measured spectrophotometrically at 540 nm using an automatic microplate reader (Eliza MAT 2000, DRG Instruments, GmbH, Marburg, Germany). Each experiment was performed two times in triplicate. Cell viability is expressed as a percentage relative to the control cells. FIG. 8 the results of the assay for composition with different ratios vitamin D3/sphingomyelin.

### Reverse Transcription Quantitative PCR (RTqPCR)

Cells were cultured for 24 h in the absence (CTR) or presence of compositions for use according to the present disclosure with different vitamin D3/sphingomyelin ratio. Total RNA was extracted from control and treated cells by using RNAqueous®-4PCR kit (Ambion Inc., Austin, Texas). Samples were treated and RTqPCR was performed according to Codini et al., “Gentamicin arrests cancer cell growth: the intriguing involvement of nuclear sphingomyelin metabolism”, Int. J. Mol. Sci. 16:2307-2319, doi: 10.3390/ijms16022307, by targeting NEFH (Hs00912472). Glyceraldehide-3-phosphate dehydrogenase (GAPDH) was used as control gene.

Results are graphically illustrated in FIG. 9.

A composition for use according to the present invention obviates the drawbacks of the prior art and brings important advantages.

An important advantage consists in the fact that a composition for use according to the present invention comprising vitamin D3 and sphingomyelin allows a considerable increase of both the length of the neurites and the volume of the cells of the nervous system in an optimum manner with respect to the prior art.

1. Use of a composition comprising vitamin D3 and sphingomyelin to promote, support or optimize the formation and outgrowth of neurites of the cells of the nervous system.

2. Use of a composition comprising vitamin D3 and sphingomyelin according to claim 1, wherein the composition comprises from 400 IU to 5000 IU of vitamin D3 and from 2 to 50 mg of sphingomyelin.

3. Use of a composition comprising vitamin D3 and sphingomyelin according to claim 1, wherein the composition comprises vitamin D3 and sphingomyelin in a ratio vitamin D3/sphingomyelin equal to or greater than 10:1 (IU/mg), preferably equal to or greater than 50:1 (IU/mg).

4. Use of a composition comprising vitamin D3 and sphingomyelin according to claim 3, wherein the ratio vitamin D3/sphingomyelin ranges from 10:1 (IU/mg) to 400:1 (IU/mg), preferably from 50:1 (IU/mg) to 400:1 (IU/mg), still more preferably from 150:1 (IU/mg) to 350:1 (IU/mg), still more preferably from 160:1 (IU/mg) to 250:1 (IU/mg).

5. Use of a composition comprising vitamin D3 and sphingomyelin according to claim 3, wherein the ratio vitamin D3/sphingomyelin is 200:1 (IU/mg).

6. Use of a composition comprising vitamin D3 and sphingomyelin according to claim 1, wherein the composition comprises 400IU of vitamin D3 and 2 mg of sphingomyelin.

7. Use of a composition comprising vitamin D3 and sphingomyelin according to claim 1, wherein the composition further comprises at least one additional component selected from the group consisting of docosahexaenoic acid (DHA), Vitamin E, medium-chain triglycerides, glycerophospholipids, ceramides, cholesterol and mixtures thereof.

8. Use of a composition comprising vitamin D3 and sphingomyelin according to claim 1, wherein the composition is for oral administration.

9. Use of a composition comprising vitamin D3 and sphingomyelin according to claim 1, wherein the composition is in a form selected from a capsule, a tablet, a water-soluble powder, a liquid solution, a hydroalcoholic solution and an edible product.

10. A method for promoting, supporting or optimizing the formation and outgrowth of neurites of the cells of the nervous system comprising the step (i) of administering to a mammalian, preferably to a human being, a composition comprising vitamin D3 and sphingomyelin.

11. The method according to claim 10, wherein the step (i) comprises orally administering the composition comprising vitamin D3 and sphingomyelin.

12. The method according to claim 11, wherein the step (i) comprises orally administering the composition comprising vitamin D3 and sphingomyelin in a form selected from a capsule, a tablet, a water-soluble powder, a liquid solution, a hydroalcoholic solution and an edible product.

13. The method according to claim 10, wherein the composition comprising from 400 IU to 5000 IU of vitamin D3 and from 2 to 50 mg of sphingomyelin.

14. The method according to claim 10, wherein the step (i) comprises administering to a mammalian, preferably to a human being, from 400IU to 5000IU of vitamin D3 and from 2 to 50 mg of sphingomyelin at least once a day.

15. A composition for oral administration comprising from 400 IU to 5000 IU of vitamin D3 and from 2 to 50 mg of sphingomyelin, wherein the ratio of vitamin D3 to sphingomyelin is equal to or greater than 50:1 (IU/mg).

16. The composition for oral administration according to claim 15, wherein the ratio vitamin D3/sphingomyelin ranges from 50:1 (IU/mg) to 400:1 (IU/mg), still more preferably from 150:1 (IU/mg) to 350:1 (IU/mg), still more preferably from 160:1 (IU/mg) to 250:1 (IU/mg).

17. The composition for oral administration according to claim 15, wherein the wherein the ratio vitamin D3/sphingomyelin is 200:1 (IU/mg).

18. The composition for oral administration according to claim 13 comprising 400IU of vitamin D3 and 2 mg of sphingomyelin.

19. The composition for oral administration according to claim 15 further comprising at least one additional component selected from the group consisting of DHA, Vitamin E, medium-chain triglycerides, glycerophospholipids, ceramides and mixtures thereof.

20. The composition for oral administration according to claim 15 in a form selected from a capsule, a tablet, a water-soluble powder, a liquid solution, a hydroalcoholic solution and an edible product.
